(19) United States
(12) Patent Application Publication
(10) Pub. No.: US 2011/0003829 A1
(43) Pub. Date: Jan. 6, 2011

(54) ENROFLOXACIN HEXAHYDRATE

(75) Inventors: Alfons Grunenberg, Dormagen (DE); Clemens Bothe, Leverkusen (DE); Birgit Keil, Dusseldorf (DE)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/524,787

(22) PCT Filed: Jan. 18, 2008
(86) PCT No.: PCT/EP08/00357

§ 371 Date: Jul. 28, 2009

(30) Foreign Application Priority Data
Jan. 31, 2007 (DE) 10 2007 004 732.2

(51) Int. Cl. A61K 31/496; C07D 401/10; A61P 31/04

(52) U.S. Cl. 514/253.08; 544/363

(57) ABSTRACT

The present invention relates to a novel hexahydrate of enrofloxacin, to processes for its preparation, to pharmaceuticals containing it, and to its use in combating diseases.

The present invention relates to a novel hexahydrate of enrofloxacin, to processes for its preparation, to pharmaceuticals containing it, and to its use in combating diseases.

The compound enrofloxacin is disclosed for example in EP-A 49 355 and EP-A 78 362 and is as defined in the Formula (I):

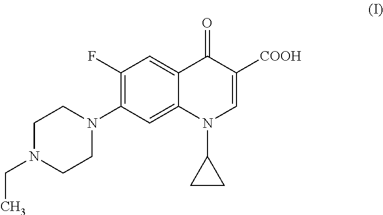

The compound of the Formula (I) is a fluoroquinolone antibiotic which is suitable for the treatment of bacterial diseases. Enrofloxacin-containing products are applied in veterinary medicine and have been commercially available for many years under the name Baytril®.

The compound of the Formula (I) can be prepared as described in EP-A 49 355 and EP-A 78 362. To date, only one crystal modification was known of the compound of the Formula (I), which is hereinbelow referred to as modification A. Modification A has a melting point of 224° C. and a characteristic x-ray diffractogram, IR spectrum, Raman spectrum, FIR spectrum and NIR spectrum (Tab. 1-5, FIG. 1-5).

Surprisingly, there has now been found a novel enrofloxacin hexahydrate of the Formula (II).

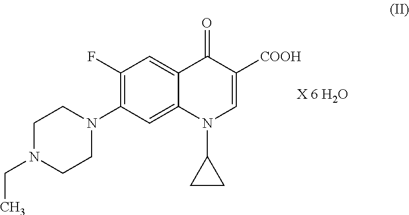

The hexahydrate of the Formula (II) contains 23.1% of hydrate water.

The present invention relates to the enrofloxacin hexahydrate of the Formula (II).

Surprisingly, the hexahydrate according to the invention shows better filtration properties and is easier to dry than modification A. Moreover, the hexahydrate according to the invention can be prepared in a better space-time yield and with a better secondary component profile than modification A.

The improved product properties are retained when the known modification A is prepared from the hexahydrate by drying.

In comparison with modification A, the hexahydrate of the Formula (II) has a clearly distinguishable X-ray diffractogram, IR spectrum, Raman spectrum, FIR spectrum and NIR spectrum (FIG. 1-5).

The invention relates in particular to enrofloxacin hexahydrate which, in the X-ray diffractogram, has a reflection at a 2 theta angle of 24.2.

The invention furthermore relates in particular to an enrofloxacin hexahydrate which, in the NIR spectrum, has a band at 5097 cm⁻¹.

The invention furthermore relates to the use of the hexahydrate of the Formula (II) for the treatment and/or prophylaxis of bacterial diseases. The enrofloxacin hexahydrate can be employed essentially for the same indications as enrofloxacin and its pharmaceutically acceptable salts.

The present invention furthermore relates to the use of the compound according to the invention for the treatment and/or prophylaxis of diseases, in particular of bacterial diseases.

The present invention furthermore relates to the use of the compound according to the invention for the preparation of a pharmaceutical for the treatment and/or prophylaxis of diseases, in particular of bacterial diseases.

The present invention furthermore relates to a method of treating bacterial diseases, where a suitable amount of enrofloxacin hexahydrate is administered.

The present invention furthermore relates to pharmaceuticals which comprise the compound according to the invention, conventionally together with one or more inert non-toxic pharmaceutically acceptable adjuvants, and to their use for the abovementioned purposes.

The present invention furthermore relates to pharmaceuticals comprising the compound according to the invention and, if appropriate, one or more further active substances, in particular for the treatment and/or prophylaxis of the abovementioned diseases.

Like enrofloxacin and its salts, enrofloxacin hexahydrate, too, shows low toxicity and is active against a broad spectrum of microorganisms, including those which are resistant to a variety of antibiotics such as, for example, penicillins, cephalosporins, aminoglycosides, sulphonamides, tetracyclins. Enrofloxacin hexahydrate can be used for controlling Gram-negative and Gram-positive bacteria and bacteria-like microorganisms, and the diseases caused by these pathogens can be prevented, alleviated and/or cured. Accordingly, the hexahydrate is suitable in human and veterinary medicine for the prophylaxis and chemotherapy of local and systemic infections which are caused by these pathogens.

It is furthermore also suitable as an agent for the preservation of inorganic and organic materials, in particular organic materials of various types, for example polymers, lubricants, colours, fibres, leather, paper and wood, of foodstuffs and of water.

The hexahydrate can be used in a variety of pharmaceutical preparations. Preferred pharmaceutical preparations which may be mentioned are tablets, including sugar-coated tablets, capsules, pills, granules, suppositories, solutions, suspensions and emulsions for injection, oral solutions, suspensions and emulsions, furthermore pastes, ointments, gels, creams, lotions, powders and sprays.

Usually, a pharmaceutical formulation will, for stability reasons, contain mainly the hexahydrate of the Formula (II) and no substantial amounts of another form such as, for example, of another modification or of a solvate of the compound of the Formula (II). The pharmaceutical preferably contains more than 90 percent by weight, especially preferably more than 95 percent by weight, of the hexahydrate of the Formula (II), based on the total amount of the compound which it contains.

Enrofloxacin hexahydrate has favourable toxicity to warm-blooded species and is preferably suitable for combating bacterial diseases which occur in animal keeping and animal breeding in productive livestock, breeding stock, zoo animals, laboratory animals, experimental animals and pets. In this context, they are active against all or individual developmental stages and against resistant and normally sensitive strains. By combating the bacterial diseases, it is intended to reduce illness, deaths and reduced performance (for example in the production of meat, milk, wool, hides, eggs, honey and the like), so that, by using the active substances, more economical and simpler animal keeping is possible. The productive livestock and breeding stock include mammals such as, for example, cattle, horses, sheep, pigs, goats, camels, water buffalos, donkeys, rabbits, fallow deer, reindeer, fur-bearing animals such as, for example, mink, chinchilla, raccoon, birds such as, for example, chickens, geese, turkeys, ducks, pigeons, bird species kept on domestic premises and in zoos. They furthermore include farmed fish and ornamental fish.

The laboratory and experimental animals include mice, rats, guinea pigs, golden hamsters, dogs and cats.

The pets include dogs and cats.

In general, it has proved advantageous to administer amounts of from approximately 0.5 to approximately 50 mg, preferably 1 to 20 mg, of active substance per kg body weight per day in order to achieve effective results. The active substances can also be administered together with the animals' feed or drinking water.

Feed and foodstuffs usually contain from 0.01 to 100 ppm, preferably from 0.5 to 50 ppm, of the active substance in combination with a suitable edible material.

Such a feeding stuff and foodstuff can be used both for curative purposes and for prophylactic purposes.

Such a feeding stuff or foodstuff is prepared by mixing, with customary feeding stuffs, a concentrate or a blend which contains from 0.5 to 30% by weight, preferably from 1 to 20% by weight of an active substance in a mixture with an edible organic or inorganic carrier. Examples of edible carriers are maize meal or maize and soybean meal or mineral salts which preferably contain a small amount of an edible dust-prevention oil, for example corn oil or soya oil. The blend thus obtained can then be added to the complete feeding stuff before it is fed to the animals.

The invention furthermore relates to a process for the preparation of the hexahydrate of the Formula (II) by dissolving the compound of the Formula (I) in modification A in an inert solvent or in solvent/water mixtures and by converting the active substance into the hexahydrate of the Formula (II) by the addition of water at a temperature of between 5° C. and 25° C., preferably of from 20 to 25° C. The precipitate is isolated and dried at room temperature. This gives the hexahydrate of the Formula (II). The identity of the hexahydrate of the Formula (II) can be verified for example by X-ray diffractometry and by thermogravimetric analysis (TGA).

The invention furthermore relates to a process for the preparation of the hexahydrate of the Formula (II) by suspending the compound of the Formula (I) in modification A in water and by converting it into the hexahydrate of the Formula (II) by stirring or shaking the suspension. The residue is isolated and dried at room temperature. The identity of the hexahydrate of the Formula (II) can be verified for example by X-ray diffractometry and by thermogravimetric analysis (TGA).

Suitable inert solvents are mainly water-miscible solvents with boiling points of up to approximately 120° C. such as, for example, lower alcohols, in particular aliphatic alcohols with one hydroxyl group and 1 to 4 carbon atoms such as, for example, methanol, ethanol, isopropanol, or other volatile solvents such as, for example acetonitrile, or mixtures of the abovementioned solvents, or mixtures of the abovementioned solvents with water. Preferred are acetonitrile, methanol and isopropanol or mixtures of the abovementioned solvents or mixtures of the abovementioned solvents with water, very especially preferably ethanol or mixtures of ethanol with water.

The hexahydrate of the Formula (II) is preferably prepared by dissolving the compound of the Formula (I) in modification A in ethanol/water (1:1) or methanol and precipitating the hexahydrate by addition of water at a temperature of between 5 and 25° C., preferably at a temperature of from 20 to 25° C. The precipitate is isolated and dried. This gives the hexahydrate of the Formula (II).

The invention furthermore relates to a process for the preparation of a purified form of enrofloxacin in modification A. Here, the hexahydrate is prepared by seeding an aqueous suspension of modification A with the hexahydrate of the Formula (II), subsequently removing the solvent and converting back the hexahydrate into modification A. This last step can be effected by drying at a higher temperature, in vacuo, at low atmospheric humidity or by stirring in anhydrous solvents such as, for example, absolute ethanol.

### USE EXAMPLES

The DSC and TGA thermograms were obtained using a Differential Scanning Calorimeter DSC 7 or Pyris-1 (heating rate 2 K/min, flushing with dry nitrogen) and a Thermogravimetric Analyser TGA 7 (heating rate 10 K/min, flushing with dry nitrogen) from Perkin-Elmer. The X-ray diffractograms were registered in a Stoe transmission diffractometer using CuKα radiation. The IR, FIR, NIR and Raman spectra were recorded with Fourier IR spectrometers IFS 66/IFS 66 v (IR) with 32 scans and a resolution of 2 cm⁻¹, IFS 66v (FIR) with 100 scans and a resolution of 2 cm⁻¹, IFS 28/N (NIR) with 15 scans and a resolution of 8 cm⁻¹ and RFS 100 (Raman) with 64 scans and a resolution of 2 cm⁻¹ from Bruker.

### Preparation of the Hexahydrate of Enrofloxacin

### Example 1

Approximately 100 mg of enrofloxacin in modification A are suspended in approximately 2 ml of water and shaken at 25° C. After 8 days, the residue is filtered off and dried at room temperature. It is analysed by X-ray diffractometry and corresponds to the title compound as hexahydrate.

### Example 2

Approximately 100 mg of enrofloxacin in modification A are dissolved with heating in approximately 10 ml of acetonitrile. The solution is filtered, treated with approximately 100 ml of water and left to stand in the refrigerator. On the next day, the active substance which has precipitated is filtered off and dried at room temperature. It is analysed by thermogravimetric analysis and corresponds to the title compound as hexahydrate.

### Example 3

Approximately 100 mg of enrofloxacin in modification A are dissolved with heating in approximately 10 ml of methanol. The solution is filtered and treated with approximately 10 ml of water. The solution is left to stand at room temperature until the solvent has evaporated. The residue is analysed by thermogravimetric analysis and corresponds to the title compound as hexahydrate.

### Example 4

Approximately 100 mg of enrofloxacin in modification A are suspended in approximately 2 ml of isopropanol:water (1:1) and shaken at 5° C. After one week, the residue is filtered off and dried at room temperature. It is analysed by thermogravimetric analysis and corresponds to the title compound as hexahydrate.

### Example 5

Approximately 4 g of enrofloxacin in modification A are suspended in approximately 80 ml of ethanol:water (1:1) and stirred at room temperature. After one week, the residue is filtered off and dried at room temperature. It is analysed by thermogravimetric analysis and corresponds to the title compound as hexahydrate.

### Example 6

Approximately 500 mg of enrofloxacin in modification A are suspended in approximately 20 ml of ethanol:water (1:1) and stirred at room temperature. After 1.5 h, the suspension is seeded with the hexahydrate. After 24 h, the residue is filtered off and dried at room temperature. It is analysed by thermogravimetric analysis and corresponds to the title compound as hexahydrate.

### Preparation of Modification a from Enrofloxacin Hexahydrate

### Example 7

100 mg of enrofloxacin hexahydrate are dried for one hour in the drying oven at 60° C. The residue is analysed by thermogravimetric analysis and corresponds to the title compound in modification A.

### Example 8

100 mg of enrofloxacin hexahydrate are suspended in approximately 2 ml of absolute ethanol and shaken at 25° C. After 24 h, the residue is filtered off and dried at room temperature. It is analysed by thermogravimetric analysis and corresponds to the title compound in modification A.

### Example 9

100 mg of enrofloxacin hexahydrate are dried for 24 h at room temperature in vacuo. The residue is analysed by thermogravimetric analysis and corresponds to the title compound in modification A.

### Example 10

100 mg of enrofloxacin hexahydrate are dried for 24 h at room temperature over phosphorus pentoxide. The residue is analysed by thermogravimetric analysis and corresponds to the title compound in modification A.

TABLE 1
X-ray diffractometry
Reflections [2 theta]

| Modification A | Hexahydrate |
|---|---|
| 7.2 | 6.9 |
| 8.7 | 7.2 |
| 9.8 | 8.1 |
| 12.6 | 9.7 |
| 13.5 | 11.7 |
| 14.5 | 13.9 |
| 14.9 | 14.3 |
| 15.3 | 14.5 |
| 16.0 | 14.6 |
| 16.5 | 14.8 |
| 17.4 | 14.9 |
| 17.9 | 15.2 |
| 18.7 | 15.9 |
| 19.3 | 16.1 |
| 19.6 | 17.4 |
| 21.3 | 18.9 |
| 21.5 | 19.3 |
| 21.7 | 19.5 |
| 22.6 | 20.1 |
| 23.4 | 20.7 |
| 24.1 | 21.4 |
| 24.7 | 21.8 |
| 25.3 | 22.5 |
| 25.8 | 22.8 |
| 26.1 | 23.3 |
| 26.8 | 24.2 |
| 27.5 | 24.7 |
| 28.4 | 24.9 |
| 29.5 | 25.2 |
| | 25.7 |
| | 26.4 |
| | 27.0 |
| | 27.3 |
| | 27.8 |
| | 30.1 |
| | 30.7 |

TABLE 2
IR spectroscopy
Peak maxima [cm⁻¹]

| Modification A | Hexahydrate |
|---|---|
| 625 | 533 |
| 639 | 547 |
| 708 | 625 |
| 749 | 707 |
| 785 | 744 |
| 803 | 788 |
| 831 | 803 |
| 855 | 824 |
| 890 | 830 |
| 935 | 844 |
| 954 | 890 |
| 1023 | 943 |
| 1044 | 951 |
| 1077 | 1012 |
| 1090 | 1025 |
| 1107 | 1042 |
| 1124 | 1090 |
| 1154 | 1107 |
| 1186 | 1130 |
| 1208 | 1165 |
| 1221 | 1182 |
| 1254 | 1256 |
| 1289 | 1294 |
| 1298 | 1311 |
| 1313 | 1339 |
| 1337 | 1360 |
| 1381 | 1378 |
| 1393 | 1387 |
| 1401 | 1394 |
| 1467 | 1471 |
| 1508 | 1496 |
| 1539 | 1547 |
| 1611 | 1582 |
| 1628 | 1628 |
| 1737 | 1738 |
| 2780 | 3394 |
| 2826 | |
| 2875 | |
| 2967 | |
| 3090 | |

TABLE 3
Raman spectroscopy
Peak maxima [cm⁻¹]

| Modification A | Hexahydrate |
|---|---|
| 112 | 85 |
| 196 | 114 |
| 207 | 200 |
| 260 | 254 |
| 292 | 299 |
| 300 | 323 |
| 362 | 372 |
| 383 | 395 |
| 442 | 496 |
| 495 | 548 |
| 538 | 637 |
| 638 | 667 |
| 666 | 701 |
| 691 | 744 |
| 711 | 775 |
| 748 | 794 |
| 771 | 830 |
| 786 | 853 |
| 890 | 891 |
| 1026 | 944 |
| 1044 | 959 |
| 1077 | 1028 |
| 1125 | 1048 |
| 1163 | 1107 |
| 1186 | 1129 |
| 1207 | 1177 |
| 1218 | 1195 |
| 1227 | 1224 |
| 1254 | 1256 |
| 1299 | 1282 |
| 1327 | 1313 |
| 1343 | 1341 |
| 1349 | 1359 |
| 1395 | 1377 |
| 1437 | 1390 |
| 1466 | 1421 |
| 1536 | 1449 |
| 1606 | 1466 |
| 1624 | 1478 |
| 1738 | 1495 |
| 2828 | 1530 |
| 2961 | 1551 |
| 3012 | 1585 |
| 3033 | 1618 |
| | 2970 |
| | 3011 |
| | 3029 |
| | 3097 |

TABLE 4
FIR spectroscopy
Peak maxima [cm⁻¹]

| Modification A | Hexahydrate |
|---|---|
| 85 | 93 |
| 102 | 97 |
| 127 | 101 |
| 154 | 109 |
| 195 | 118 |
| 236 | 145 |
| 257 | 151 |
| 303 | 188 |
| 313 | 190 |
| 322 | 236 |
| 336 | 247 |
| 364 | 254 |
| 386 | 280 |
| 394 | 303 |
| 410 | 322 |
| 423 | 338 |
| 444 | 368 |
| 463 | 387 |
| 473 | 395 |
| 493 | 410 |
| | 423 |
| | 459 |
| | 472 |
| | 475 |
| | 495 |

TABLE 5
NIR spectroscopy
Peak maxima [cm⁻¹]

| Modification A | Hexahydrate |
|---|---|
| 4041 | 4049 |
| 4087 | 4129 |
| 4123 | 4212 |
| 4192 | 4277 |
| 4216 | 4341 |
| 4254 | 4383 |
| 4330 | 4433 |
| 4390 | 4501 |
| 4490 | 4553 |
| 4543 | 5097 |
| 4950 | 5857 |
| 5236 | 5943 |
| 5660 | 5980 |
| 5792 | 6053 |
| 5947 | 6133 |
| 6039 | 6162 |
| 6109 | 6679 |
| 7165 | 8515 |
| 7986 | 8788 |
| 8433 | |
| 8733 | |

### FIGURES

FIG. 1: X-ray diffractograms of enrofloxacin modification A and hexahydrate

FIG. 2: Infrared spectra of enrofloxacin modification A and hexahydrate

FIG. 3: Raman spectra of enrofloxacin modification A and hexahydrate

FIG. 4: FIR spectra of enrofloxacin modification A and hexahydrate

FIG. 5: NIR spectra of enrofloxacin modification A and hexahydrate

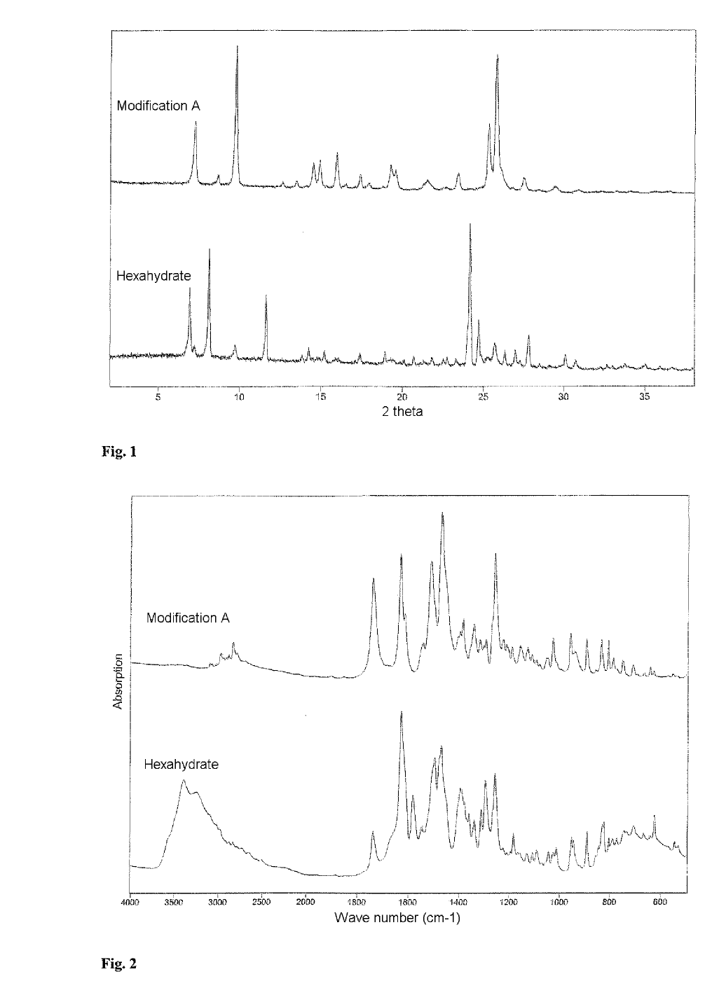

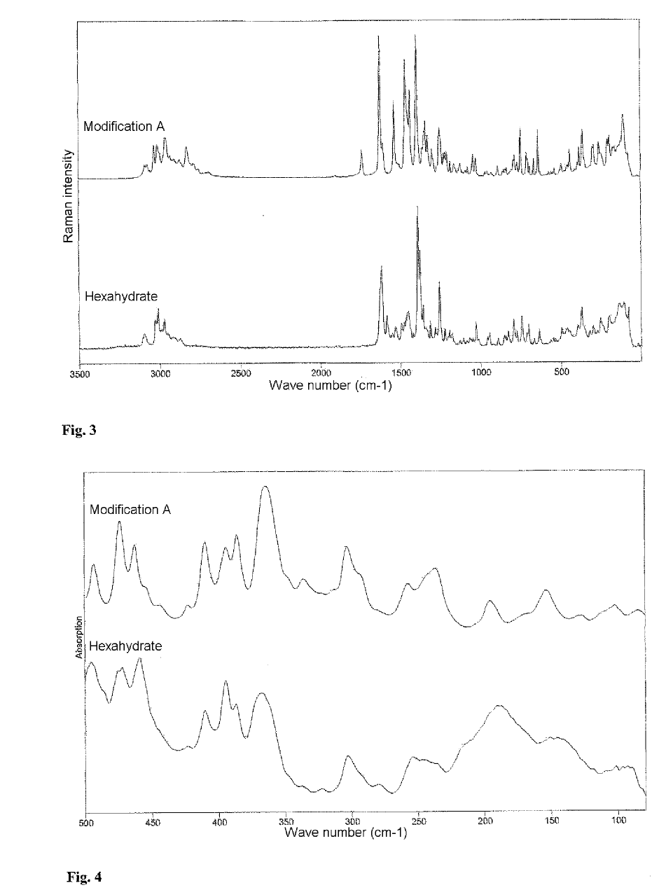

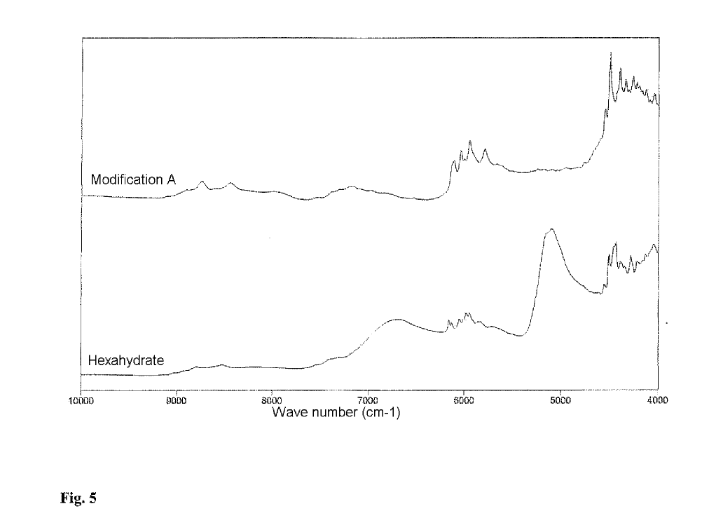

1. An enrofloxacin hexahydrate compound, of the Formula (II)

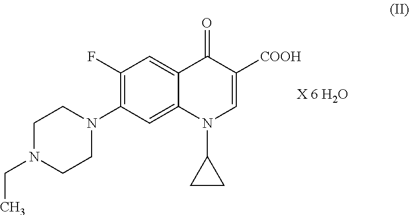

2. The compound of claim 1 which, in the X-ray diffractogram, has a reflection at a 2 theta angle of 24.2.

3. The compound of claim 1 which, in the NIR spectrum, has a band at 5097 cm 1.

4. A pharmaceutical composition comprising enrofloxacin hexahydrate according to claim 1.

5. (canceled)

6. A method of treating bacterial diseases in an animal, which comprises administering an effective amount of enrofloxacin hexahydrate to an animal in need thereof

7. (canceled)
